(19) United States
(12) Patent Application Publication
(10) Pub. No.: US 2024/0112340 A1
(43) Pub. Date: Apr. 4, 2024

(54) INFORMATION PROCESSING APPARATUS, RADIATION IMAGING SYSTEM, INFORMATION PROCESSING METHOD, AND NON-TRANSITORY COMPUTER-READABLE STORAGE MEDIUM

(71) Applicant: CANON KABUSHIKI KAISHA, Tokyo (JP)

(72) Inventor: Hiroyuki OMI, Kanagawa (JP)

(21) Appl. No.: 18/470,457

(22) Filed: Sep. 20, 2023

(30) Foreign Application Priority Data
Sep. 27, 2022 (JP) 2022-154001

(51) Int. Cl. G06T 7/00; A61B 6/00

(52) U.S. Cl. CPC G06T 7/0014; A61B 6/505; A61B 6/582; G06T 2207/10116; G06T 2207/30008

(57) ABSTRACT

An information processing apparatus includes: a first obtaining unit configured to, using data obtained by imaging a first object with known bone information via radiation irradiation based on a first imaging condition, obtain calibration data of the bone information; and a correction unit configured to, using the calibration data, correct bone information of a second object different from the first object in a case where a comparison result of the first imaging condition and a second imaging condition satisfies a predetermined condition, the bone information being obtained using data obtained by imaging the second object via radiation irradiation based on the second imaging condition.

### BACKGROUND OF THE INVENTION

### Field of the Invention

The techniques of the disclosure relate to an information processing apparatus, a radiation imaging system, an information processing method, and a non-transitory computer-readable storage medium.

### Description of the Related Art

A known quantification method for bone mineral in bone includes the Dual-energy X-ray Absorptiometry (DXA) method (hereinafter, referred to as the “DXA method”) of using two X-rays with different energy distributions to measure the bone density from the difference in X-ray absorption coefficient between soft tissue and bone tissue. With a bone density measurement apparatus using the DXA method, a line sensor is used to irradiate X-rays on a line by line basis and obtain data. Thus, a significant amount of time is required for one instance of imaging, which places a burden on the patient, that is, the object.

Recently, digital image diagnosis using an X-ray image captured using a flat panel sensor (hereinafter, referred to as the “sensor”) has become more common and is also being used in bone density measurement. When the sensor is used in bone density imaging, X-rays irradiate the entire sensor surface (cone beam imaging) to obtain an image. Accordingly, the time taken for one instance of imaging is reduced, and the imaging is less of a burden for the patient.

The method for increasing bone density measurement accuracy described in Japanese Patent Laid-Open No. 2021-037164 includes using machine learning to correctly extract a region, such as the lumbar spine or the femur, corresponding to the bone density measurement target. Also, the method described in Japanese Patent Laid-Open No. 2018-192054 includes analyzing correction data to prevent a reduction in the measurement accuracy due to degradation of the sensor over time. The disclosure provides techniques for enhancing information obtaining accuracy.

### SUMMARY OF THE INVENTION

According to one aspect of the present invention, there is provided an information processing apparatus comprising: a first obtaining unit configured to, using data obtained by imaging a first object with known bone information via radiation irradiation based on a first imaging condition, obtain calibration data of the bone information; and a correction unit configured to, using the calibration data, correct bone information of a second object different from the first object in a case where a comparison result of the first imaging condition and a second imaging condition satisfies a predetermined condition, the bone information of the second object being obtained using data obtained by imaging the second object via radiation irradiation based on the second imaging condition.

According to another aspect of the present invention, there is provided an information processing method comprising: using data obtained by imaging a first object with known bone information via radiation irradiation based on a first imaging condition, obtaining calibration data of the bone information; and using the calibration data, correcting bone information of a second object different from the first object in a case where a comparison result of the first imaging condition and a second imaging condition satisfies a predetermined condition, the bone information of the second object being obtained using data obtained by imaging the second object via radiation irradiation based on the second imaging condition.

Further features of the present invention will become apparent from the following description of exemplary embodiments with reference to the attached drawings.

### BRIEF DESCRIPTION OF THE DRAWINGS

FIG. 1 is a diagram illustrating the configuration of a radiation imaging system according to an embodiment.

FIG. 2 is a diagram illustrating an example of the hardware configuration of the radiation imaging system.

FIG. 3 is a flowchart illustrating the overall processing process of a radiation imaging system according to a first embodiment.

FIG. 4 is a diagram for describing an irradiation field region.

FIG. 5 is a flowchart illustrating the processing process of processing for obtaining bone information calibration data.

FIG. 6 is a diagram for describing an example of the processing for obtaining bone information calibration data.

FIG. 7 is a diagram schematically illustrating a region where two irradiation field regions overlap.

FIG. 8 is a diagram for describing a threshold of comparison information.

FIG. 9 is a diagram illustrating an example notification of a message.

FIG. 10 is a flowchart illustrating the overall processing process of a radiation imaging system according to a second embodiment.

FIG. 11 is a diagram for describing an irradiation field region according to the second embodiment.

FIG. 12 is a flowchart illustrating the overall processing process of a radiation imaging system according to a third embodiment.

FIG. 13 is a diagram illustrating the configuration of a radiation imaging system according to a fourth embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

To calculate the bone density from an image obtained by the sensor, calibration between a value obtained from the sensor output value and the actual bone density value is required. By imaging a bone density calibration phantom (Quality Control phantom, hereinafter, referred to as the “QC phantom”) with a known bone density, calibration data of bone information, which is a value for bone density calibration, can be obtained. By calibrating the value obtained from the sensor output value with the calibration data, the bone density is obtained.

The effects of scattered rays can cause a reduction in measurement accuracy. For example, when the sensor is used to perform bone density imaging and cone beam imaging is used, the output value of the sensor changes due to the effects of scattered rays, making the bone density also fluctuate. Thus, methods are used for reducing these effects including using a lattice grid or narrowing with a collimator. Accordingly, the same imaging conditions are desirably used when imaging the object and when imaging the QC phantom.

When measuring bone density, there are many imaging conditions, such as imaging distance, tube voltage, dose, collimator open/close state, and the like, that often need to be set manually. Thus, when object imaging and QC phantom imaging are performed with different imaging conditions, the accuracy of the bone density (hereinafter also referred to as the “bone information”) may be reduced. Regarding this, an information processing apparatus and a radiation imaging system according to the present embodiment described below are designed to enhance bone information obtaining accuracy.

Radiation according to the techniques of the present disclosure includes α-rays, (β-rays, and γ-rays, which are beams of particles (including photons) emitted due to radioactive decay, as well as beams with approximately equal or greater energy, such as X-rays, a particle beam, and cosmic rays.

### First Embodiment

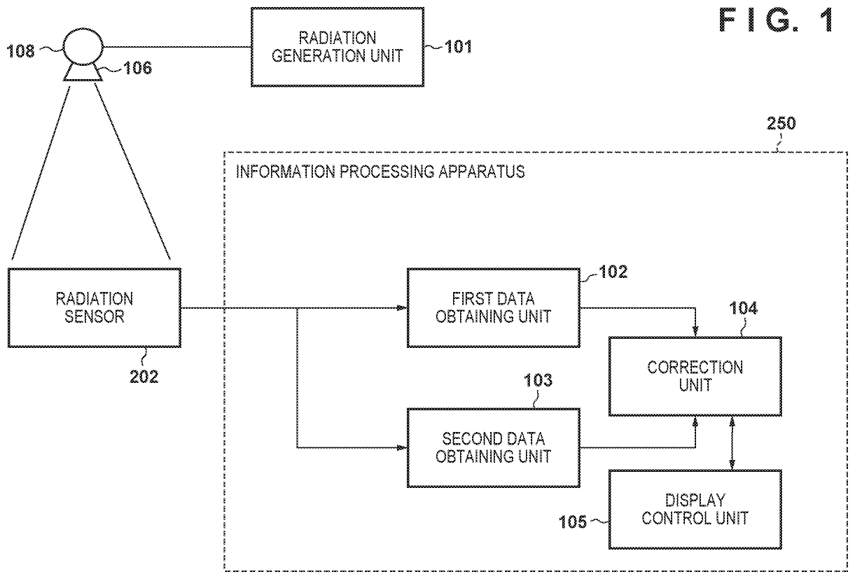

FIG. 1 is a diagram illustrating the configuration of a radiation imaging system according to the first embodiment. The radiation imaging system includes a radiation generation unit 101, a radiation detection apparatus (hereinafter referred to as the “radiation sensor 202”), and an information processing apparatus 250 (a first data obtaining unit 102, a second data obtaining unit 103, and a correction unit 104).

The information processing apparatus 250 includes, as a functional configuration, the first data obtaining unit 102, the second data obtaining unit 103, the correction unit 104, and a display control unit 105. This functional configuration, for example, is implemented by one or more Central Processing Units (CPUs) executing a program read out from a storage unit. The configuration of the units of the information processing apparatus 250 may include integrated circuits and the like, as long as a similar function is achieved. Also, the information processing apparatus 250 may include, as an internal configuration, a graphics control unit such as a Graphics Processing Unit (GPU) and a communication unit such as a network card or the like.

The radiation generation unit 101 generates radiation using the specified imaging conditions. When an exposure switch is pressed by an operator, the radiation generation unit 101 generates a high voltage pulse in a radiation tube 108 to generate radiation, and the radiation tube 108 emits radiation. At this time, a collimator 106 can be used to narrow the radiation irradiation range to prevent the radiation from being emitted outside of the region of interest of the object. This allows unnecessary exposure to be reduced and scattered rays generated from the object to be reduced.

The radiation sensor 202 is constituted by a radiation Flat Panel Detector (FPD), for example. In the radiation sensor 202, a phosphor (scintillator) that converts detected radiation into light and a photoelectric conversion element that outputs a signal corresponding to the converted light are provided for each pixel disposed in an array (two-dimensional region). The photoelectric conversion elements of the pixels convert radiation converted into visible light by the phosphors into a detection signal, and the detection signal is output to the information processing apparatus 250.

The first data obtaining unit 102 uses the detection signal (detection data) of the radiation sensor 202 having imaged, via radiation irradiation based on a first imaging condition, a first object (“bone density calibration phantom” or “QC phantom”) with known bone information to obtain calibration data. Here, “calibration data” is data for converting bone information (bone density) dens obtained by imaging the first object (QC phantom) with known bone information into actual bone information specified by the QC phantom.

The second data obtaining unit 103 uses the detection signal (detection data) of the radiation sensor 202 having imaged, via radiation irradiation based on a second imaging condition, a second object (hereinafter also referred to as the “object (patient)” different from the first object to obtain an irradiation field region (second irradiation field region) via radiation irradiation based on the second imaging condition.

When the comparison result between the first imaging condition and the second imaging condition satisfies a predetermined condition, the correction unit 104 corrects the bone information of the second object using the calibration data.

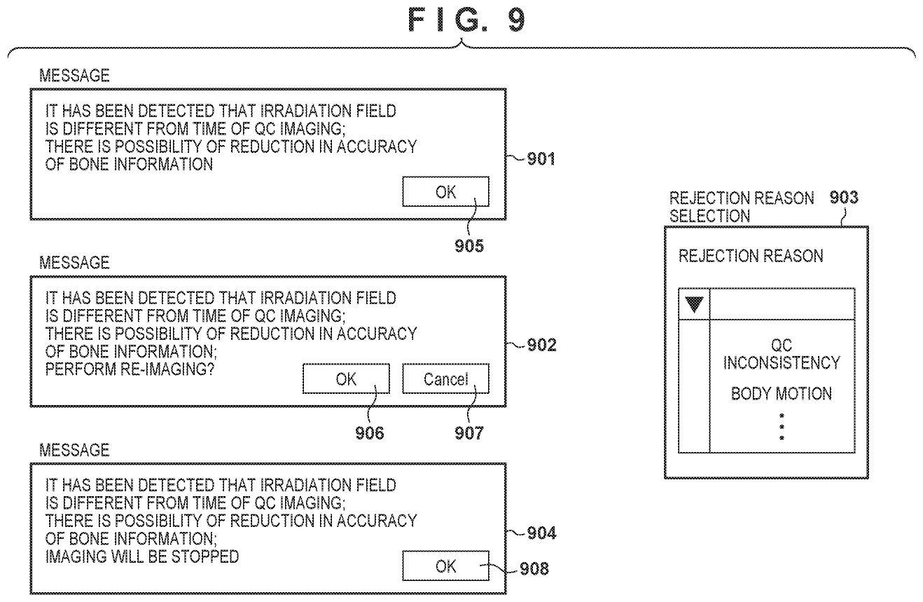

Also, the correction unit 104 obtains comparison information via the comparison between the first imaging condition and the second imaging condition, and the display control unit 105 performs display control to display a message (see FIG. 9 for example) based on the comparison information obtained by the correction unit 104 on display units (205, 212, 213).

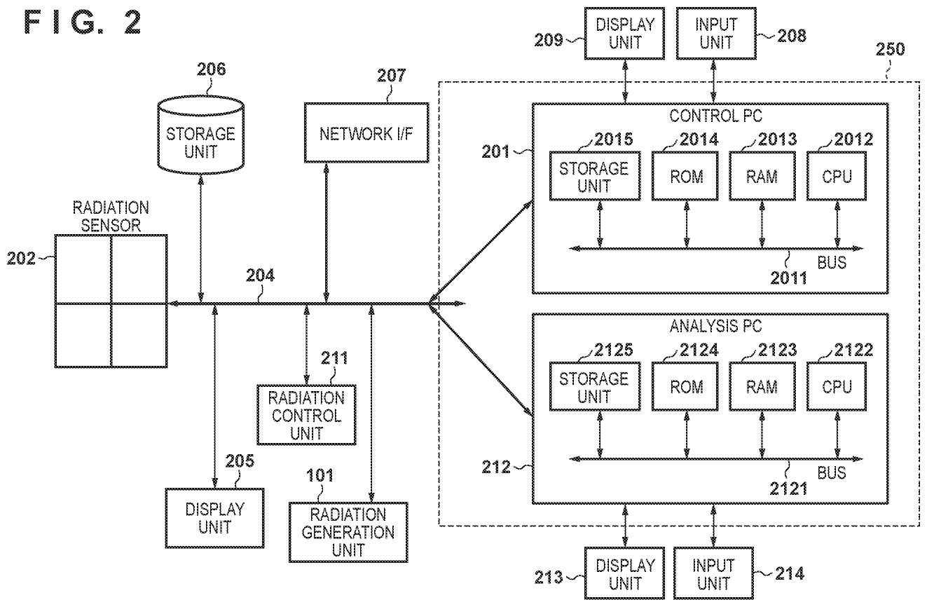

FIG. 2 is a diagram illustrating an example of the hardware configuration of the radiation imaging system. The hardware configuration in FIG. 2 is obtained by implementing the configuration in FIG. 1 using hardware, for example.

The information processing apparatus 250 includes a first processing unit (hereinafter referred to as a control PC 201) and a second processing unit (hereinafter referred to as an analysis PC 212) that function as processing units.

The control PC 201 and the analysis PC 212 and the radiation sensor 202 are connected via a communication line 204 and the like of a Gigabit Ethernet (registered trademark) or the like. Also, the radiation generation unit 101, the display unit 205, a storage unit 206, and a network interface unit 207, and a radiation control unit 211 that controls the radiation generation unit 101 are connected by the communication line 204. Note that for the communication line 204 for connection, other than Gigabit Ethernet (registered trademark), for example, Controller Area Network (CAN), optical fiber, or the like can be used.

An input unit 208 is connected to the control PC 201 via Universal Serial Bus (USB) or Personal System/2 (PS/2) that functions as an interface. Also, a display unit 209 is connected via DisplayPort of Digital Visual Interface (DVI). Commands are sent to the radiation sensor 202, the display unit 205, and the like via the control PC 201.

In the internal configuration of the control PC 201, for example, a Central Processing Unit (CPU) 2012, a Random Access Memory (RAM) 2013, a Read Only Memory (ROM) 2014, and a storage unit 2015 are connected via a bus 2011.

The software module relating to the processing contents for each imaging mode are stored in the ROM 2014 or the storage unit 2015. The software module instructed by a non-illustrated instruction unit is loaded onto the RAM 2013 and executed by the CPU 2012.

The detection signal (data) obtained by the radiation sensor 202 is sent to the storage unit 2015 inside the control PC 201 or the storage unit 206 outside the control PC 201 and stored.

In the internal configuration of the analysis PC 212 connected to the communication line 204, for example, a Central Processing Unit (CPU) 2122, a Random Access Memory (RAM) 2123, a Read Only Memory (ROM) 2124, and a storage unit 2125 are connected via a bus 2121. Also, an input unit 214 is connected to the analysis PC 212 via Universal Serial Bus (USB) or Personal System/2 (PS/2) that functions as an interface. Also, a display unit 213 is connected via DisplayPort of Digital Visual Interface (DVI).

In the analysis PC 212, software modules relating to the processing content of the bone information obtaining (bone density calculation), the creation content of the bone information analysis (bone density analysis) report, and the creation content of a message based on the comparison information obtained by the correction unit 104 are stored in the ROM 2124 or the storage unit 2125. The software module instructed by a non-illustrated instruction unit is loaded onto the RAM 2123 and executed by the CPU 2122. The processed image and the created report are sent to the storage unit 2125 inside the analysis PC 212 or the storage unit 206 outside the analysis PC 212 and stored.

The functional configuration (the first data obtaining unit 102, the second data obtaining unit 103, the correction unit 104, and the display control unit 105) described with reference to FIG. 1 are stored in the ROM 2014 and the storage unit 2015 or the ROM 2124 and the storage unit 2125. The functional configuration of the first data obtaining unit 102, the second data obtaining unit 103, the correction unit 104, and the display control unit 105 may be mounted as a dedicated information processing board or optimally mounted depending on the purpose.

The processing of the first data obtaining unit 102, the second data obtaining unit 103, the correction unit 104, and the display control unit 105 in the radiation imaging system provided with the information processing apparatus 250 with the configuration described above will be described below in detail.

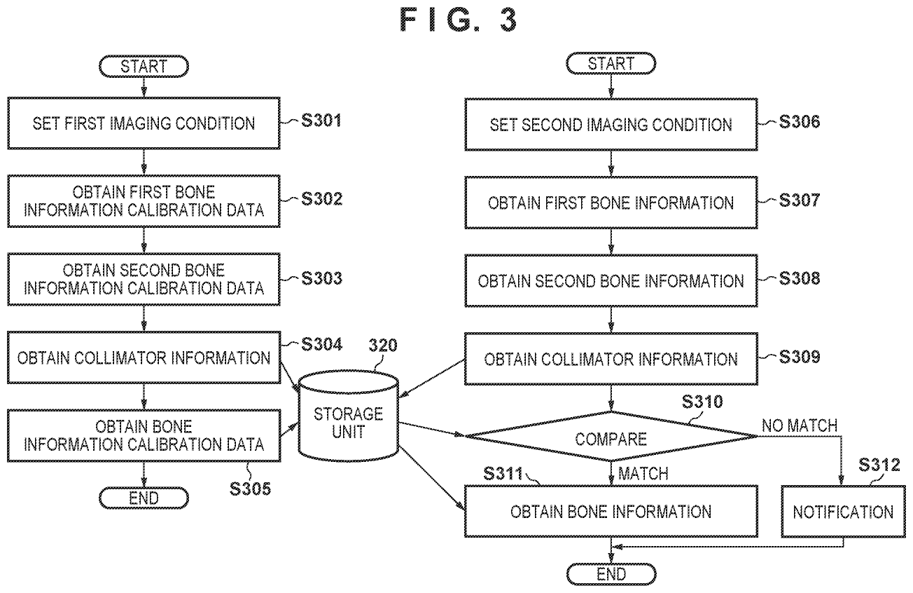

FIG. 3 is a flowchart illustrating the overall processing process of the radiation imaging system according to the first embodiment. The overall flow of the processing in the radiation imaging system will be described using the configuration of the radiation imaging system described with reference to FIG. 1 and the flowchart illustrating the overall processing process illustrated in FIG. 3.

### Step S301: Setting the First Imaging Condition

In step S301, the first imaging condition is set in the radiation generation unit 101. Here, the first imaging condition includes, for example, the tube current, the irradiation duration, the tube voltage, and other radiation generation conditions and the irradiation angle at the radiation sensor 202, Source to Image Distance (SID), collimator 106 open/close state, presence of grid, and other geometric conditions. Here, SID indicates the distance between the radiation tube 108 and the radiation sensor 202.

To obtain the detection signal (data) when imaging with different radiation energies, the radiation generation condition includes a radiation generation condition for obtaining first energy data (low energy data) and a radiation generation condition for obtaining second energy data (high energy data) with energy higher than the first energy.

By performing sampling a plurality of times relative to one instance of radiation irradiation, the radiation sensor 202 can obtain a detection signal (low energy data) via low energy radiation and a detection signal (high energy data) via high energy radiation with one instance of radiation irradiation.

### Step S302: Obtaining the First Bone Information (Bone Density) Calibration Data

In step S302, with the QC phantom placed as the object, the radiation generation unit 101 generates radiation on the basis of the set first imaging condition. The first data obtaining unit 102 obtains the calibration data of the bone information (bone density) on the basis of the detection signals of the radiation sensor 202 obtained by imaging the QC phantom using radiation generated using the first imaging condition. In the present step, the first data obtaining unit 102 (first obtaining unit) obtains the calibration data of the first bone information using data obtained by imaging the QC phantom (first object) using radiation of the first energy.

### Step S303: Obtaining the Second Bone Information (Bone Density) Calibration Data

In step S303, the first data obtaining unit 102 obtains the calibration data of the bone information (bone density) on the basis of the detection signals of the radiation sensor 202 obtained by imaging the QC phantom using radiation generated using the first imaging condition. In the present step, the first data obtaining unit 102 obtains the calibration data of the second bone information using data obtained by imaging the QC phantom (first object) using radiation of the second energy higher than the first energy.

### Step S304: Obtaining the Collimator Information

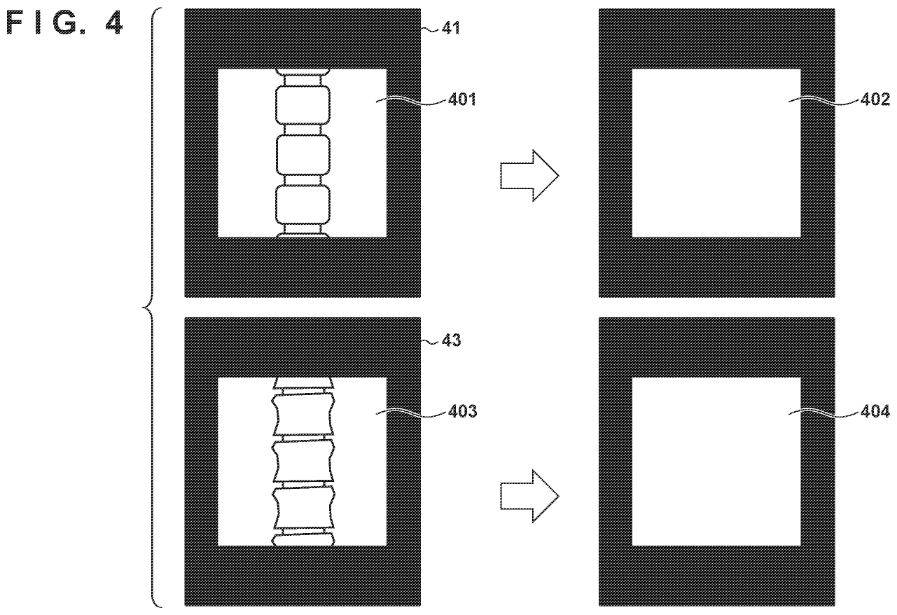

In step S304, the first data obtaining unit 102 obtains the irradiation field region (hereinafter also referred to as the “first irradiation field region”) of the radiation irradiated using the first imaging condition as the collimator information from the calibration data of the first bone information or the calibration data of the second bone information. In FIG. 4, an irradiation field region 401 indicates an irradiation field region in a radiation image 41 obtained by imaging the first object (QC phantom). An irradiation field region 402 indicates an irradiation field region obtained by applying a rulebase or a trained machine learning technique to the radiation image 41.

As the irradiation field recognition method for obtaining the irradiation field region 402, for example, an image-processing-based rulebase or a trained machine learning technique is used. Processing using Hough transform can be used as the method using a rulebase, for example. Using Hough transform, the linear components included in the image data can be extracted. By narrowing down the linear component using pixel value and geometric arrangement conditions, the first data obtaining unit 102 can obtain the irradiation field region 402.

Semantic segmentation to segment the image into discretionary regions can be used as the method using trained machine learning, for example. A trained model for implementing semantic segmentation can be obtained via machine learning (deep learning). By using machine learning using a learning model such as SegNET, U-NET, or the like for the machine learning, trained data for recognizing irradiation fields can be obtained. By performing machine learning using the irradiation field region data as the correct data, a trained machine learning model for implementing semantic segmentation to segment the irradiation field region can be obtained.

Also, the first data obtaining unit 102 may use both a rulebase and machine learning to obtain an irradiation field region obtained via radiation irradiation based on the first imaging condition. For example, a known technique using machine learning to recognize roughly the irradiation field region and thereafter increase the accuracy via a rulebase. The first data obtaining unit 102 may obtain the irradiation field region using this technique.

The first data obtaining unit 102 stores the obtained irradiation field region 402 in a storage unit 320. The recognized irradiation field region may be two-dimensional image information as with the irradiation field region 402, but may instead be coordinate information. The storage unit 320 illustrated in FIG. 3 represents the storage units 206, 2015, and 2125 described with reference to FIG. 2, and the storage destination of the result of irradiation field recognition may be any one of the storage units 206, 2015, or 2125.

### Step S305: Obtaining the Bone Information (Bone Density) Calibration Data

In step S305, the first data obtaining unit 102 obtains the calibration data of the bone information (bone density) from the calibration data of the first bone information (bone density) and the calibration data of the second bone information (bone density) and stores the calibration data of the bone information (bone density) in the storage unit 320.

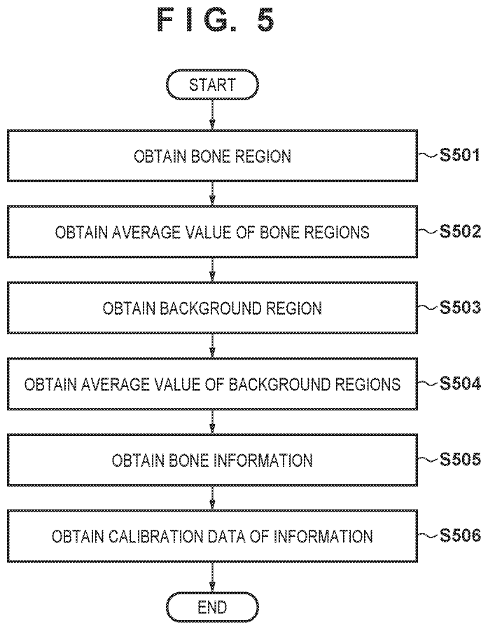

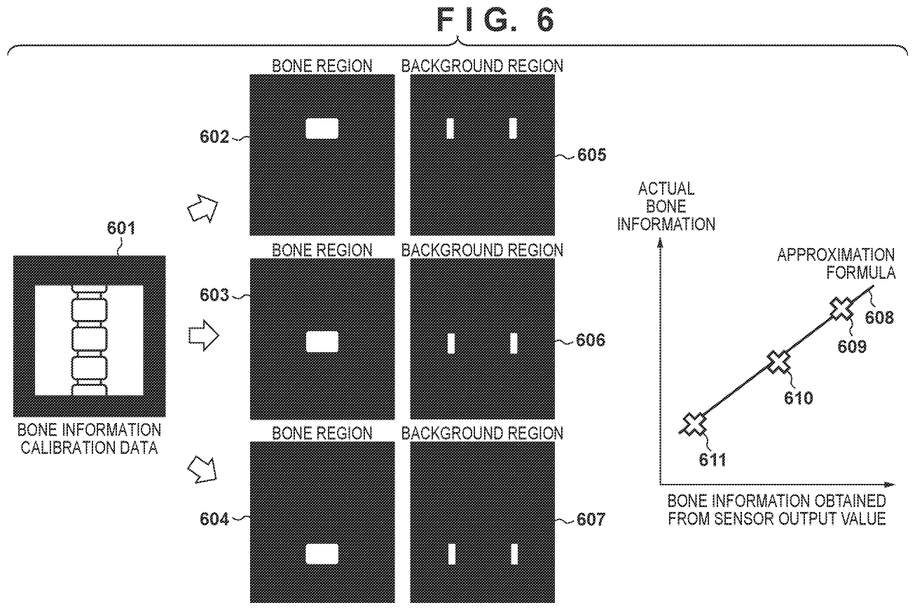

FIG. 5 is a flowchart illustrating the processing process of processing for obtaining bone information calibration data. FIG. 6 is a diagram for describing an example of the processing for obtaining bone information calibration data. A specific processing process of processing for obtaining bone information calibration data will be described below using the flowchart in FIG. 5 and FIG. 6.

### Step S501: Obtaining the Bone Region

In step S501, from calibration data 601 of the bone information (bone density), the first data obtaining unit 102 obtains the bone region with the vertebral bodies included in the QC phantom sorted. Here, for the calibration data 601 of the bone information (bone density) to be used to obtain a bone region with the vertebral bodies sorted, the calibration data of the first bone information or the calibration data of the second bone information may be used. In the example illustrated in FIG. 6, the vertebral bodies are sorted into a plurality of types (three types, for example), and the first data obtaining unit 102 obtains a bone region 602, a bone region 603, and a bone region 604 as regions with the vertebral bodies sorted.

For the first data obtaining unit 102 to obtain the regions (bone regions 602 to 604) with the vertebral bodies sorted into a plurality of types, for example, an image-processing-based rulebase or a trained machine learning technique is used.

Otsu's method can be used as the method using a rulebase, for example. Otsu's method is a method of maximizing inter-class variance, and thus can segment the bone region from other regions. By narrowing down the geometric information, the bone region 602, the bone region 603, and the bone region 604 can be obtained.

Semantic segmentation can be used as the method using machine learning, for example. A trained model for implementing semantic segmentation can be obtained via machine learning (deep learning). By using machine learning using a learning model such as SegNET, U-NET, or the like for the machine learning, trained data for sorting vertebral bodies into type can be obtained. By performing machine learning using data with the vertebral bodies sorted into type as the correct data as a learning set, a trained machine learning model for implementing semantic segmentation to segment the bone regions by vertebral body type can be obtained. Also, the first data obtaining unit 102 may use both a rulebase and machine learning together to obtain the regions (bone regions) with the vertebral bodies sorted into a plurality of types. The first data obtaining unit 102 obtains and outputs, as a bone region obtaining result, a bone region mask in which “1” is set for the identification information indicating a bone region and “0” is set for the identification information indicating regions other than a bone region.

### Step S502: Obtaining the Average Value of Bone Regions

In step S502, the first data obtaining unit 102 obtains an average value boValue based on the difference between the calibration data of the second bone information for the bone regions and the calibration data of the first bone information. The first data obtaining unit 102 obtains the average value (hereinafter also referred to as the “bone region average value”) based on the difference between the calibration data of the second bone information for the bone regions and the calibration data of the first bone information using Mathematical Formula 1.

$$bo Value ( i ) = \frac{1}{nbo} {\sum }_{x} {\sum }_{y} [ log ( HImg ( x , y ) ) - α \cdot log ( LImg ( x , y ) ) ] \cdot Bo Mask ( i , x , y ) \qquad Math . 1$$

Here, LImg represents the calibration data (low energy data) of the first bone information, and HImg represents the calibration data (high energy data) of the second bone information. BoMaSk represents the bone region mask, nbo represents the number of pixels where the bone region mask is 1, α represents the difference coefficient, and i represents the number of obtained bone regions. In the present embodiment, since there are three bone regions, the average value (bone region average value) is obtained from the three bone regions.

### Step S503: Obtaining the Background Region

In step S503, the first data obtaining unit 102 obtains a background region 605, a background region 606, and a background region 607 for each of the bone region 602, the bone region 603, and the bone region 604 with the bone information (bone density) sorted from the calibration data 601. Here, a background region indicates a soft tissue region without a bone region. Also, for the calibration data 601 of the bone information (bone density) to be used to obtain a background region, the calibration data of the first bone information or the calibration data of the second bone information may be used.

For the first data obtaining unit 102 to obtain a background region, for example, a rulebase or a trained machine learning technique is used. A method of providing a region of a fixed size at a region a certain distance away from the bone region obtained by the processing of step S501 may be used as the method using a rulebase.

Also, as the method using machine learning, background regions may be obtained in a similar manner to the bone regions. The first data obtaining unit 102 obtains and outputs, as a background region obtaining result, a background region mask in which “1” is set for the identification information indicating a background region and “0” is set for the identification information indicating regions other than a background region.

### Step S504: Obtaining the Average Value of Background Regions

In step S504, the first data obtaining unit 102 obtains an average value bgValue based on the difference between the calibration data of the second bone information for the background regions and the calibration data of the first bone information. The first data obtaining unit 102 obtains the average value (hereinafter also referred to as the “background region average value”) based on the difference between the calibration data of the second bone information for the background regions and the calibration data of the first bone information using Mathematical Formula 2.

$$bg Value ( i ) = \frac{1}{nbg} {\sum }_{x} {\sum }_{y} [ log ( HImg ( x , y ) ) - α \cdot log ( LImg ( x , y ) ) ] \cdot Bg Mask ( i , x , y ) \qquad Math . 2$$

Here, LImg represents the calibration data (low energy data) of the first bone information, and HImg represents the calibration data (high energy data) of the second bone information. BgMaSk represents the background region mask, nbg represents the number of pixels where the background region mask is 1, α represents the difference coefficient, and i represents the number of extracted bone regions. In the present embodiment, since there are three background regions, the average value is obtained from the three background regions.

### Step S505: Obtaining the Bone Information (Bone Density)

In step S505, the first data obtaining unit 102 obtains bone information (bone density) dens obtained by imaging the first object (QC phantom) from Mathematical Formula 3 using a bone region average value (boValue(i)) and a background region average value (bgValue(i)). In the present embodiment, since there are three bone regions, three pieces of bone information (bone density) are obtained. In Mathematical Formula 3, i represents the number of obtained bone regions. The first data obtaining unit 102 obtains the bone information in the bone region of the first object (QC phantom) obtained from the difference (Mathematical Formula 3) between the background region average value and the bone region average value.

dens(i)=bgValue(i)−boValue(i) Math. 3

### Step S506: Obtaining the Corrected Bone Information (Bone Density)

The first data obtaining unit 102 compares the bone information (bone density) dens obtained in step S505 and the bone information (bone density) specified in the QC phantom and obtains a calibration value (bone information calibration data) for converting the bone information (bone density) dens obtained in step S505 into the actual bone information (bone density).

The first data obtaining unit 102 obtains the bone information calibration data by converting the bone information in the bone region of the QC phantom (first object) into known bone information. The first data obtaining unit 102 obtains the bone information calibration data on the basis of an Approximation Formula 608 obtained by the method of least squares using the bone information in the bone region of the QC phantom (first object) and the known bone information.

In the graph of the Approximation Formula 608, the horizontal axis represents the bone information obtained from the output value (sensor output value) of the radiation sensor 202, and the vertical axis represents the known bone information (actual bone information) of the QC phantom. Bone information 609 represents the bone information in the bone region 602. Also, bone information 610 represents the bone information in the bone region 603, and bone information 611 represents the bone information in the bone region 604.

The bone information (bone density) of each vertebral body in the QC phantom is known in advance, and the known bone information (bone density) is set as the actual bone information (bone density). Via the processing from step S501 to step S505, the Approximation Formula 608 is obtained via the method of least squares from the bone information (bone density) and the actual bone information (bone density), and coefficients a₀, a₁ of the obtained Approximation Formula 608 are taken as the bone information calibration data. The first data obtaining unit 102 stores the calibration data obtained via the present steps in the storage unit 320.

### Step S306: Setting the Second Imaging Condition

Returning to step S306 in FIG. 3, in step S306, the second imaging condition is set for the radiation generation unit 101. Here, the second imaging condition includes, for example, the tube current, the irradiation duration, the tube voltage, and other radiation generation conditions and the irradiation angle at the radiation sensor 202, SID, collimator 106 open/close state, presence of grid, and other geometric conditions.

To obtain the detection signal (data) when imaging with different radiation energies, the radiation generation condition includes a radiation generation condition for obtaining first energy data (low energy data) and a radiation generation condition for obtaining second energy data (high energy data) with energy higher than the first energy.

### Step S307: Obtaining the First Bone Information (Bone Density)

In step S307, with the patient placed as the object, the radiation generation unit 101 generates radiation on the basis of the set second imaging condition. The second data obtaining unit 103 obtains the bone information (bone density) on the basis of the detection signals of the radiation sensor 202 obtained by imaging the object (patient) using radiation generated using the second imaging condition. In the present step, the second data obtaining unit 103 obtains the first bone information (bone density) using data obtained by imaging the second object (object (patient)) using radiation of the first energy.

### Step S308: Obtaining the Second Bone Information (Bone Density)

In step S308, the second data obtaining unit 103 obtains the bone information (bone density) on the basis of the detection signals of the radiation sensor 202 obtained by imaging the object (patient) using radiation generated using the second imaging condition. In the present step, the second data obtaining unit 103 obtains the second bone information (bone density) using data obtained by imaging the second object (object (patient)) using radiation of the second energy higher than the first energy.

### Step S309: Obtaining the Collimator Information

In step S309, the second data obtaining unit 103 obtains the irradiation field region (hereinafter also referred to as the “second irradiation field region”) of the radiation irradiated using the second imaging condition as the collimator information from the first bone information (bone density) or the second bone information (bone density). An irradiation field region 403 in FIG. 4 indicates an irradiation field region in a radiation image 43 obtained by imaging the second object (patient). An irradiation field region 404 indicates an irradiation field region obtained by applying a rulebase or a trained machine learning technique to the radiation image 43. The method for obtaining the irradiation field region 404 is similar to that processing method of step S304. The second data obtaining unit 103 stores the obtained irradiation field region 404 in a storage unit 320.

The irradiation field region recognition result may be two-dimensional image information as with the irradiation field region 404, but may instead be coordinate information. The storage unit 320 illustrated in FIG. 3 represents the storage units 206, 2015, and 2125 described with reference to FIG. 2, and the storage destination of the result of irradiation field recognition may be any one of the storage units 206, 2015, or 2125.

### Step S310: Comparing the Comparison Information and Threshold

In step S310, the correction unit 104 obtains comparison information indicating the result of comparing the first imaging condition and the second imaging condition and compares the comparison information and a threshold. An example of a comparison between the first imaging condition and the second imaging condition includes the correction unit 104, using area as a comparison parameter, obtaining the overlapping ratio of the two irradiation field regions obtained in steps S304 and S309 and comparing this to a threshold.

The correction unit 104 obtains the comparison information indicating the overlapping ratio of the two irradiation field regions 402 and 404 by comparing the irradiation field region 402 obtained by imaging the QC phantom and the irradiation field region 404 obtained by imaging the object (patient). The correction unit 104 obtains the irradiation field region 402 obtained in step S304 and the irradiation field region 404 obtained in step S309 from the storage unit 320 and compares the two irradiation field regions. Then, the correction unit 104 obtains the comparison information indicating the overlapping ratio of the two irradiation field regions. The correction unit 104 may use, as a parameter, any one of the area of the first irradiation field region (irradiation field region 402) and the second irradiation field region (irradiation field region 404), the coordinate information (for example, coordinate information of the four corners of the outline) of the irradiation field regions, and the coordinate information of the centroid of the irradiation field regions to obtain the comparison information.

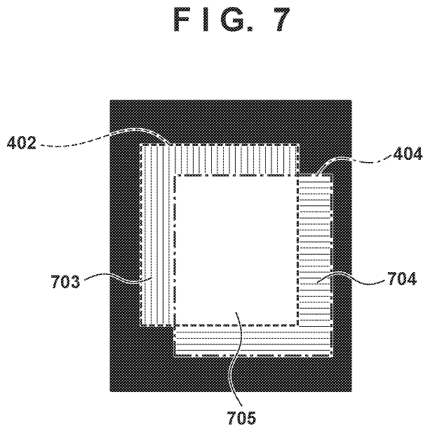

FIG. 7 is a diagram schematically illustrating the overlap of two irradiation field regions. In FIG. 7, the irradiation field regions 402 and 404 correspond to the irradiation field regions described with reference to FIG. 4. An overlap region 705 is a region where the irradiation field region 402 and the irradiation field region 404 overlap.

The region of only the irradiation field region 402 where the irradiation field region 404 does not overlap with the irradiation field region 402 corresponds to a no-match region 703 and this area is represented by Sf₁. The region of only the irradiation field region 404 where the irradiation field region 402 does not overlap with the irradiation field region 404 corresponds to a no-match region 704 and this area is represented by S_{f2}. The correction unit 104 obtains comparison information M₁ and M₂ using Mathematical Formula 4, where S₁ is the area of the irradiation field region 402.

In Mathematical Formula 4, False negative comparison information M₁ and False positive comparison information M₂ are individually defined. When the irradiation field region 404 is obtained by imaging the object (patient) using the irradiation field region 402 (area S₁) as a reference, the overlap region 705 is the region where the irradiation field region 404 corresponds with the irradiation field region 402. The region not obtained but normally required to be obtained is the no-match region 703 (false negative). The region obtained but normally not obtained is the no-match region 704 (false positive).

Math. 4

M₁=1−S_{f1}/S₁

M₂=1−S_{f2}/S₁ Math. 4

The correction unit 104 obtains first comparison information (M₁) from an area ratio for the first irradiation field region (402) obtained using the area (S_{f1}) of the first no-match region (703) not overlapped with the second irradiation field region (404) and the area (S₁) of the first irradiation field region.

Also, the correction unit 104 obtains second comparison information (M₂) from an area ratio for the second irradiation field region (404) obtained using the area (S_{f2}) of the second no-match region (704) not overlapped with the first irradiation field region (402) and the area (S₁) of the first irradiation field region.

When the no-match regions 703 and 704 are made smaller, the value of the comparison information approaches 1 and the overlapping ratio of the two irradiation field regions (irradiation field mask) increases. A state in which the comparison information is 1 indicates a state in which the first irradiation field region (402) and the second irradiation field region (404) overlap without the no-match regions 703 and 704. S_{f1} and S_{f2} may be set as a combined comparison information using Mathematical Formula 4. The area S₁ of the irradiation field region 402 is set as the denominator because, to obtain the comparison information using the information obtained by imaging the QC phantom as a reference, the area S₁ of the irradiation field region 402 is set as the denominator. Note that the comparison information is not limited to the Mathematical Formula 4 example, and the area S2 of the irradiation field region 404 may be used as the reference.

The correction unit 104 compares the obtained comparison information and the threshold of the comparison information prepared in advance and determines whether or not the comparison information exceeds the threshold.

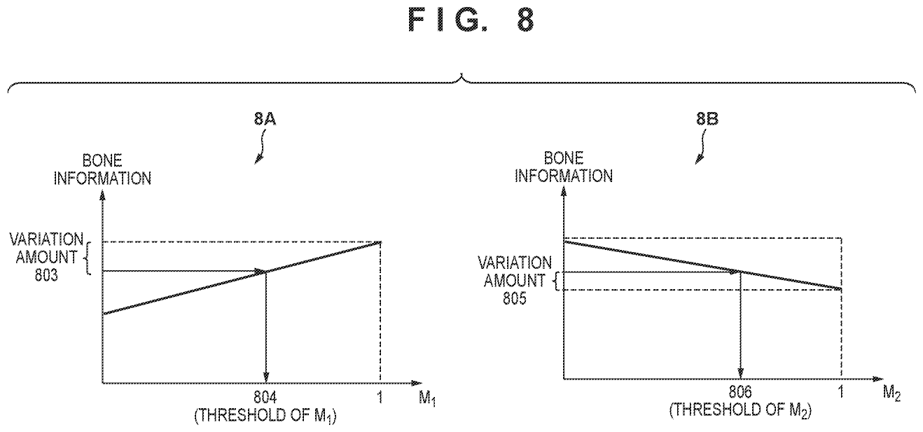

FIG. 8 is a diagram for describing the threshold of the comparison information. The threshold of the comparison information is a value set on the basis of the variation amount in the bone information (bone density) as illustrated in FIG. 8(8A and 8B). For example, the variation amount in the bone information (bone density) is measured by imaging the QC phantom while changing the degree of opening of the collimator 106.

The correction unit 104 obtains the threshold based on the variation amount in the bone information according to the degree of opening of the collimator 106. The correction unit 104 obtains, as a first threshold (804), the comparison information of when the variation amount in the bone information obtained when imaging while the degree of opening of the collimator 106 is changed in the closed direction is equal to or greater than a specific variation amount. Also, the correction unit 104 obtains, as a second threshold (806), the comparison information of when the variation amount in the bone information obtained when imaging while the degree of opening of the collimator 106 is changed in the open direction is equal to or greater than a specific variation amount.

In 8A of FIG. 8, the variation amount in the bone information (bone density) is measured while reducing the value indicating the comparison information in the closed direction of the collimator 106. From a state (comparison information M₁=1) in which the two irradiation field regions (402, 404) match, when the variation amount in the bone information (bone density) is equal to or greater than a specific value (variation amount 803), the comparison information 804 at this time is set as the threshold (first threshold) of the comparison information M₁.

In 8B of FIG. 8, the variation amount in the bone information (bone density) is measured while reducing the value indicating the comparison information in the open direction of the collimator 106. From a state (comparison information M₂=1) in which the two irradiation field regions (402, 404) match, when the variation amount in the bone information (bone density) is equal to or greater than a specific value (variation amount 805), the comparison information 806 at this time is set as the threshold (second threshold) of the comparison information M₂. The threshold is not limited to one, and a plurality of thresholds can be set. Depending on the imaging condition, this can be changed via the system settings. In this manner, a threshold appropriate for the comparison information can be appropriately set.

The correction unit 104 compares the comparison information M₁ obtained using Mathematical Formula 4 and the threshold (804). Also, the correction unit 104 compares the comparison information M₂ obtained using Mathematical Formula 4 and the threshold (806). When the first comparison information (M₁) is equal to or greater than the first threshold (804) and the second comparison information (M₂) is equal to or greater than the second threshold (806), the correction unit 104 proceeds the processing to step S311.

### Step S311: Obtaining the Bone Information (Bone Density)

In step S311, the correction unit 104 corrects the bone information of the second object (patient) using the calibration data. The correction unit 104 executes processing similar to that from step S501 to step S505 to obtain the bone information (bone density) of the object (patient). In other words, the bone region is obtained using either the first bone information (step S307) or the second bone information (step S308) obtained on the basis of the detection signals of the radiation sensor 202 obtained by imaging the object (patient) using radiation generated using the second imaging condition (step S501). Then, the correction unit 104 obtains the average value boValue (bone region average value) based on the difference between the second bone information of the bone region and the first bone information using Mathematical Formula 1 (step S502).

The correction unit 104 obtains the background region for each bone region obtained in step S501 (step S503). Then, the correction unit 104 obtains the average value bgValue (background region average value) based on the difference between the second bone information of the background region and the first bone information using Mathematical Formula 2.

The correction unit 104 obtains the bone information (bone density) dens in the bone region of the second object (patient) obtained from the difference (Mathematical Formula 3) between the background region average value (bgValue(i)) and the bone region average value (boValue(i)) (step S505).

The bone information (bone density) dens obtained using Mathematical Formula 3 is the bone information before correction via the calibration data, and the correction unit 104 obtains the bone information (bone density) dens as temporary bone information (bone density).

The correction unit 104 obtains corrected bone information (bone density) densA using the following Mathematical Formula 5 using the calibration values (bone information calibration data) a₀ and a₁ obtained in step S506 on the temporary bone information (bone density) dens.

densA=α₀·dens+α₁ Math. 5

On the other hand, when the comparison information of at least one of the comparison information M₁ and the comparison information M₂ is less than the threshold in the determination of step S310 (no match in step S310), the processing proceeds to step S312.

### Step S312: Notification

In step S312, the display control unit 105 causes the display units (205, 212, and 213) to display messages 901 and 902 notifying the user of the possibility that bone information (bone density) of a predetermined accuracy cannot be obtained. When the first comparison information M₁ is less than the first threshold (804) or the second comparison information M₂ is less than the second threshold (806), the display control unit 105 causes the display units (205, 212, and 213) to display messages notifying the user of the comparison result.

FIG. 9 is a diagram illustrating examples of the notifications of messages from the display control unit 105. The display control unit 105 causes the display units (205, 212, and 213) to display the comparison results from the correction unit 104. The display control unit 105 causes the display units (205, 212, and 213) to display the messages 901 and 902 that notify of the possibility of a reduction in the accuracy of the bone information as the comparison result.

An example of a notification method includes notifying the user of the possibility of a reduction in the accuracy of the bone information (bone density) via the message 901 in FIG. 9. The display control unit 105 causes the display units (205, 212, and 213) to display the message 901 and the bone information (bone density) densA obtained by processing similar to that in step S311.

The display control unit 105 causes the display units (205, 212, and 213) to display, in the message 901, a confirmation input interface 905 for the user to input confirmation.

The display control unit 105 causes the display units (205, 212, and 213) to display the message 901 and in addition, as with the message 902, a message for communicating the possibility of a reduction in the accuracy of the bone information (bone density) and for confirming whether re-imaging will be performed or not.

The display control unit 105 causes the display units (205, 212, and 213) to display an instruction input interface 906 for instructing to perform re-imaging, an instruction input interface 907 for cancelling re-imaging, and a rejection reason selection menu 903 for inputting the reason for re-imaging when issuing the re-imaging instruction.

When the user operates the instruction input interface 906 and selects re-imaging, the imaging is treated as a rejection and the rejection reason needs to be input. The display control unit 105 performs control to cause the display units (205, 212, and 213) to display the rejection reason selection menu 903 and allow the user to select the rejection reason relating to the imaging from among selection items including QC inconsistency, body motion, insufficient bone information (bone density) accuracy, and the like. When re-imaging is performed, re-imaging is transitioned to without obtaining the bone information (bone density) densA.

On the other hand, when re-imaging is not performed, as with the message 901, the display control unit 105 causes the display units (205, 212, and 213) to display the message 902 and the bone information (bone density) densA obtained by processing similar to that in step S311.

In the present embodiment described above, in step S310, as an example of comparing the first imaging condition and the second imaging condition, the correction unit 104 executes processing to obtain the comparison information on the basis of the areas of the irradiation field regions obtained in steps S304 and S309. However, comparison information may be compared using a comparison parameter other than the areas of the irradiation field regions. For example, the offset amount of the coordinate information of the centroids of the irradiation field regions may be used, or the offset amount of the coordinate information of the irradiation field regions (for example, the coordinate information of the four corners of the outline) may be used. Also, the size or aspect ratio of the irradiation fields may be used, or how many inches are imaged may be used.

Also, in the present embodiment described above, the threshold of the comparison information is obtained from the variation amount of the bone information (bone density) but it may be set based on an error in the degree of opening display of the collimator 106. There is a permissible tolerance between the scale for adjusting the collimator 106 and the actual degree of opening, and an offset within this tolerance can be determined a match.

In the present embodiment described above, the radiation generation condition changes between the low energy data obtaining condition and the high energy data obtaining condition. Thus, the calibration data of the first bone information and the calibration data of the second bone information and the first bone data and the second bone data are obtained.

The radiation generation condition is not limited to this example, and, for example, using the same radiation generation condition, the low energy data and the high energy data may be obtained and separated using a structure on the sensor side. For example, the radiation sensor 202 may have a multilayer structure. With such a multilayered radiation sensor 202, low energy radiation passes through the upper layer radiation sensor 202 and is detected, the radiation after passing through the upper layer radiation sensor 202 hardens, and high energy radiation is detected at the lower layer radiation sensor 202. Accordingly, a radiation image of the low energy can be obtained by the upper layer radiation sensor 202, and a radiation image of the high energy can be obtained by the lower layer radiation sensor 202.

Also, in obtaining the calibration data of the bone information, a set of low energy data and high energy data is obtained, but the dose may be changed and a plurality of sets of low energy data and high energy data may be obtained. At this time, the processing according to the present embodiment is executed with the same conditions for the dose when obtaining the calibration data of the bone information and for the dose when obtaining the bone information (bone density) of the subject (patient). According to the configuration of the present embodiment, the bone information obtaining accuracy can be improved.

### Second Embodiment

In the second embodiment, for a plurality of irradiation field regions with different sizes obtained from images of multiple instances of imaging, the first data obtaining unit 102 obtains calibration data of bone information, and the correction unit 104 corrects the bone information using the calibration data of the bone information obtained for the irradiation field region with the highest comparison information from among the plurality of irradiation field regions obtained via comparison of comparison information using the plurality of irradiation field regions and the second irradiation field region.

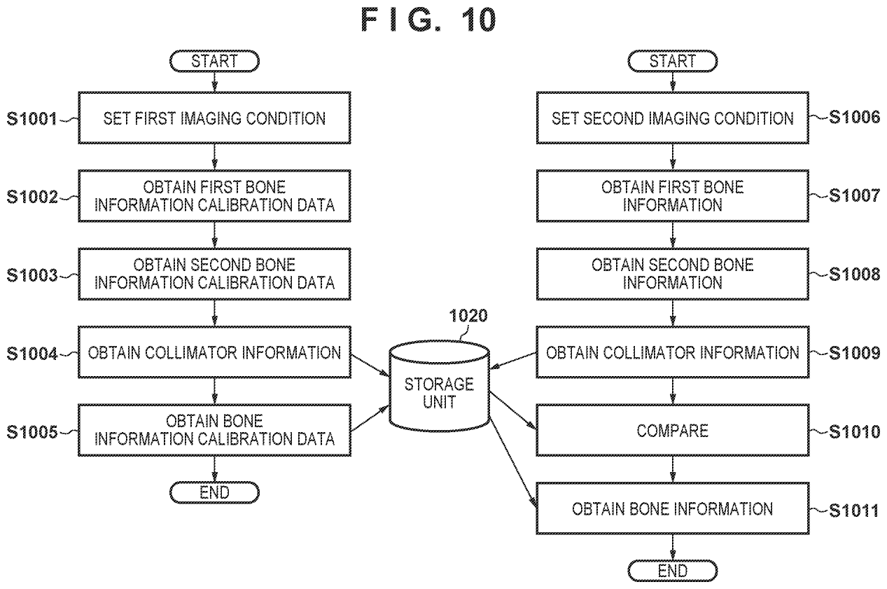

FIG. 10 is a flowchart illustrating the overall processing process of a radiation imaging system according to the second embodiment. The overall flow of the processing in the radiation imaging system will be described using the configuration of the radiation imaging system described with reference to FIG. 1 and the flowchart illustrating the overall processing process illustrated in FIG. 10. Note that in the processing process in FIG. 10, the processing from step S1001 to step S1003 is similar to the processing (from step S301 to step S303) described in the first embodiment and will thus not be described.

### Step S1004: Obtaining the Collimator Information

In step S1004, the first data obtaining unit 102 obtains the irradiation field region of the radiation irradiated using the first imaging condition from the calibration data of the first bone information or the calibration data of the second bone information. The first data obtaining unit 102 stores the obtained irradiation field region in a storage unit 1020. Here, the method for obtaining the irradiation field region is similar to the processing (step S304) described in the first embodiment.

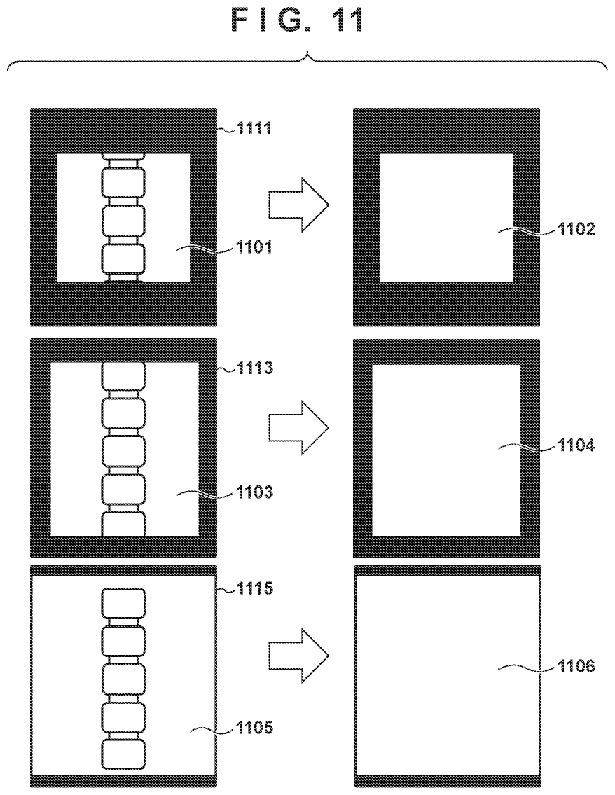

FIG. 11 is a diagram for describing the irradiation field region according to the second embodiment. In the second embodiment, imaging of the QC phantom is performed a plurality of times with different irradiation field regions. For example, the irradiation field size is changed to 9 inch, 12 inch, and 14 inch in the plurality of instances of imaging. Then, the first data obtaining unit 102 obtains an irradiation field region from each one of a plurality of radiation images 1111, 1113, and 1115 obtained from the plurality of instances of imaging.

In FIG. 11, an irradiation field region 1101 indicates an irradiation field region in a radiation image 1111 obtained by imaging the first object (QC phantom). An irradiation field region 1102 indicates an irradiation field region obtained by applying a rulebase or a trained machine learning technique to the radiation image 1111.

An irradiation field region 1103 indicates an irradiation field region in a radiation image 1113 obtained by imaging the first object (QC phantom). An irradiation field region 1104 indicates an irradiation field region obtained by applying a rulebase or a trained machine learning technique to the radiation image 1113.

In a similar manner, an irradiation field region 1105 indicates an irradiation field region in a radiation image 1115 obtained by imaging the first object (QC phantom). An irradiation field region 1106 indicates an irradiation field region obtained by applying a rulebase or a trained machine learning technique to the radiation image 1115.

The first data obtaining unit 102 stores the obtained plurality of irradiation field regions 1102, 1104, and 1106 in the storage unit 1020. Here, the storage unit 1020 illustrated in FIG. 10 represents the storage units 206, 2015, and 2125 described with reference to FIG. 2, and the storage destination of the result of obtaining the irradiation field regions may be any one of the storage units 206, 2015, or 2125.

### Step S1005: Obtaining the Bone Information (Bone Density) Calibration Data

In step S1005, the first data obtaining unit 102 obtains the calibration data of the bone information from the calibration data of the first bone information and the calibration data of the second bone information and stores the calibration data of the bone information in the storage unit 1020. In the second embodiment, the calibration data of bone information from each of the images obtained from the plurality of instances of imaging is obtained and stored in the storage unit 1020. The processing for obtaining the calibration data of the bone information is similar to the processing described with reference to FIGS. 5 and 6.

In the processing process in FIG. 10, the processing from step S1006 to step S1008 is similar to the processing (from step S306 to step S308) described in the first embodiment and will thus not be described.

### Step S1009: Obtaining the Collimator Information

In step S1009, the second data obtaining unit 103 obtains the irradiation field region (second irradiation field region) of the radiation irradiated using the second imaging condition as the collimator information from the first bone information (bone density) or the second bone information (bone density). The method for obtaining the irradiation field region is similar to that processing method of step S304 according to the first embodiment. The second data obtaining unit 103 stores the obtained irradiation field region in a storage unit 1020.

### Step S1010: Comparing the Comparison Information and Threshold

In step S1010, the correction unit 104 compares the irradiation field region (step S1009) obtained on the basis of imaging using the second imaging condition and the plurality of irradiation field regions 1102, 1104, and 1106 (step S1004) obtained on the basis of imaging using the first imaging condition obtained from the storage unit 1020 and obtains the overlapping ratio of the irradiation field regions as the comparison information (step S1010). The correction unit 104 obtains the comparison information using Mathematical Formula 4 as described in the first embodiment. Comparison processing of the comparison information and the threshold and notification processing (step S312) for a message when the comparison information is less than the threshold are similar to those in the first embodiment. When the comparison information is equal to or greater than the threshold, the correction unit 104 proceeds the processing to step S1011.

### Step S1011: Obtaining the Bone Information (Bone Density)

In step S1011, the correction unit 104 obtains the bone information densA (Mathematical Formula 5) obtained by correcting the temporary bone information dens (Mathematical Formula 3) using the calibration data of the bone information corresponding to the irradiation field region with the highest comparison information from among the irradiation field masks 1102, 1104, and 1106.

Also, the correction unit 104 may obtain a plurality of pieces of bone information using the calibration data of the bone information obtained for a plurality of irradiation field regions selected in order from highest comparison information or may obtain bone information obtained via interpolation of a plurality of pieces of bone information using weighting according to the comparison information. For example, the correction unit 104 can use the calibration data of bone information corresponding to the top two irradiation field regions, from among the irradiation field regions 1102, 1104, and 1106, in terms of highest comparison information with the irradiation field region (step S1009) obtained on the basis of imaging using the second imaging condition. The correction unit 104 may obtain two pieces of the bone information (bone density) dens (Mathematical Formula 3) using the calibration data of the bone information corresponding to two irradiation field regions and obtain bone information obtained via interpolation of the two pieces of bone information (bone density) using a weight coefficient from weighting according to the comparison information.

### Third Embodiment

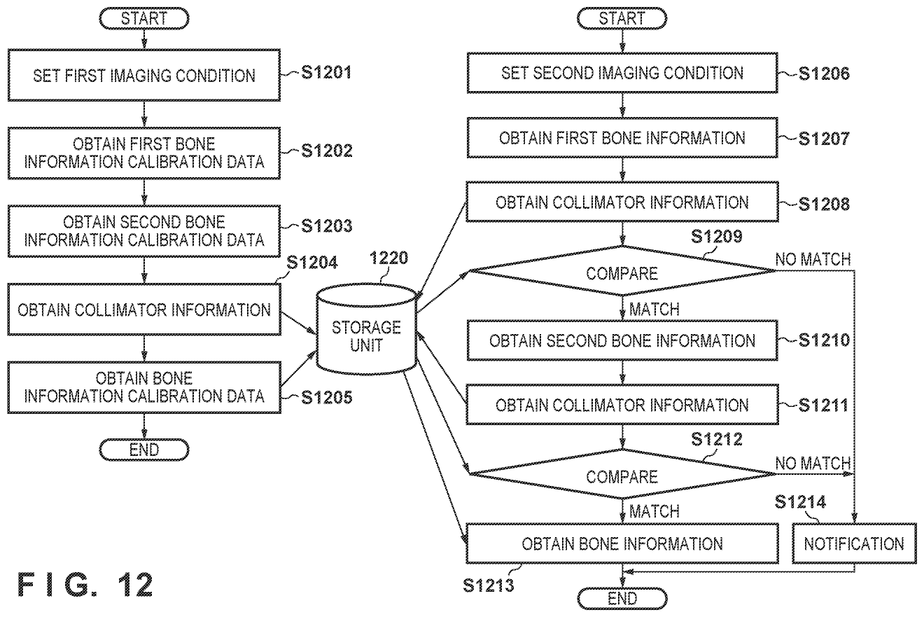

FIG. 12 is a flowchart illustrating the overall processing process of a radiation imaging system according to the third embodiment. The overall flow of the processing in the radiation imaging system will be described using the configuration of the radiation imaging system described with reference to FIG. 1 and the flowchart illustrating the overall processing process illustrated in FIG. 12. Note that in the processing process in FIG. 12, the processing from step S1201 to step S1205 is similar to the processing (from step S301 to step S305) described in the first embodiment and will thus not be described.

### Step S1206: Setting the Second Imaging Condition

In step S1206, the second imaging condition is set in the radiation generation unit 101. Here, the second imaging condition includes the tube current, the irradiation duration, the tube voltage, and other radiation generation conditions and the irradiation angle at the radiation sensor 202, SID, collimator 106 open/close state, presence of grid, and other geometric conditions.

To obtain the detection signal (data) when imaging with different radiation energies, the radiation generation condition includes a radiation generation condition for obtaining first energy data (low energy data) and a radiation generation condition for obtaining second energy data (high energy data) with energy higher than the first energy.

### Step S1207: Obtaining the First Bone Information (Bone Density)

In step S1207, with the patient placed as the object, the radiation generation unit 101 generates radiation on the basis of the set second imaging condition. The second data obtaining unit 103 obtains the bone information (bone density) on the basis of the detection signals of the radiation sensor 202 obtained by imaging the object (patient) using radiation generated using the second imaging condition. In the present step, the bone information (bone density) obtained using the radiation generation condition, from among the radiation generation conditions, for obtaining the first energy data (low energy data) is obtained as the first bone information (bone density).

### Step S1208: Obtaining the Collimator Information

In step S1208, the second data obtaining unit 103 obtains the irradiation field region (second irradiation field region) of the radiation irradiated using the second imaging condition from the first bone information (bone density). The method for obtaining the irradiation field region is similar to the processing step S309 (step S304) described in the first embodiment. The second data obtaining unit 103 stores the obtained irradiation field region in a storage unit 1220. The storage unit 1220 illustrated in FIG. 12 represents the storage units 206, 2015, and 2125 described with reference to FIG. 2, and the storage destination of the result of obtaining the irradiation field regions may be any one of the storage units 206, 2015, or 2125.

### Step S1209: Comparing the Comparison Information and Threshold

In step S1209, the correction unit 104, from the storage unit 1220, obtains the irradiation field region 402 (first irradiation field region) obtained by imaging the QC phantom and the irradiation field region 404 (second irradiation field region) obtained by imaging the object (patient) and obtains the comparison information indicating the overlapping ratio of the two irradiation field regions. The method for obtaining the comparison information is similar to the method in the first embodiment and includes obtaining the comparison information using Mathematical Formula 4.

The correction unit 104 compares the comparison information M₁ obtained using Mathematical Formula 4 and the threshold (804). Also, the correction unit 104 compares the comparison information M₂ obtained using Mathematical Formula 4 and the threshold (806). When at least one piece of comparison information from among the comparison information M₁ and the comparison information M₂ is less than the threshold (no match in step S1209), the correction unit 104 stops obtaining the high energy data due to the possibility of a reduced bone information (bone density) obtaining accuracy and stops imaging.

The display control unit 105 causes the display units (205, 212, and 213) to display a message 904 notifying the user that imaging will stop due to the bone information (bone density) of a predetermined accuracy being unable to be obtained. The display control unit 105 causes the display units (205, 212, and 213) to display, in the message 904, a confirmation input interface 908 for the user to input confirmation.

In the other hand, in comparison of step S1209, when the first comparison information (M₁) is equal to or greater than the first threshold (804) and the second comparison information (M₂) is equal to or greater than the second threshold (806) (a match in step S1209), the correction unit 104 proceeds the processing to step S1210.

### Step S1210: Obtaining the Second Bone Information (Bone Density)

In step S1210, the second data obtaining unit 103 obtains the bone information (bone density) on the basis of the detection signals of the radiation sensor 202 obtained by imaging the object (patient) using radiation generated using the second imaging condition. In the present step, the bone information (bone density) obtained using the radiation generation condition, from among the radiation generation conditions, for obtaining the second energy data (high energy data) is obtained as the second bone information (bone density).

The following processing from step S1211 to step S1214 is similar to the processing (step S309 to step S312) in the first embodiment and thus will not be described.

### Fourth Embodiment

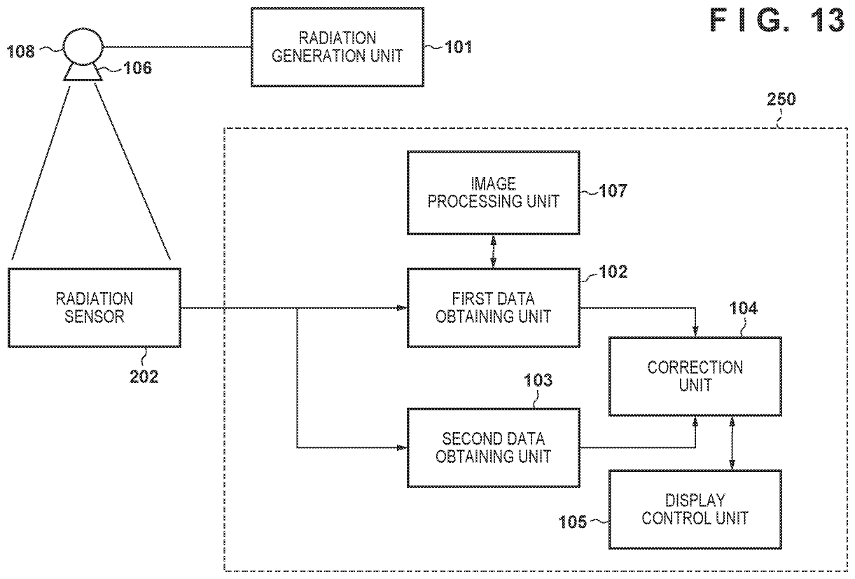

FIG. 13 is a diagram illustrating the configuration of a radiation imaging system according to the fourth embodiment. The difference from the configuration illustrated in FIG. 1 is that an image processing unit 107 is added. The functional configuration of the image processing unit 107 is one or more Central Processing Units (CPUs), and the function of the image processing unit 107 is configured by using a program read out from a storage unit. The image processing unit 107 executes image processing for reducing the scattered ray components included in the calibration data (step S302) of the first bone information and the calibration data (step S303) of the second bone information. In the configuration illustrated in FIG. 13, the radiation generation unit 101, the collimator 106, the radiation tube 108, the radiation sensor 202, and the first data obtaining unit 102, the second data obtaining unit 103, the correction unit 104, and the display control unit 105 included in the information processing apparatus 250 have a similar configuration to the configuration in FIG. 1.

For the processing of the radiation imaging system according to the fourth embodiment, the overall flow of the processing in the radiation imaging system will be described using the configuration of the radiation imaging system described with reference to FIGS. 1 and 13 and the flowchart illustrating the overall processing process illustrated in FIG. 3.

The processing from step S301 to step S304 in FIG. 3 is similar to the processing described in the first embodiment and will thus not be described.

### Step S305: Obtaining the Bone Information (Bone Density) Calibration Data

In step S305, the image processing unit 107 executes image processing on the calibration data of the first bone information obtained in step S302 and the calibration data of the second bone information obtained in step S303.

Image processing refers to the preprocessing of the calibration data of the first bone information obtained in step S302 and the calibration data of the second bone information obtained in step S303. The image processing unit 107 executes scattered ray reduction processing as the preprocessing, for example. By executing scattered ray reduction processing, the scattered ray components included in the calibration data of the bone information (calibration data of the first bone information and the calibration data of the second bone information) are reduced. The obtained value for the calibration data (of the bone density) can be changed depending on whether or not (ON or OFF) the scattered ray reduction processing is executed. Thus, according to whether the scattered ray reduction processing is ON or OFF, the calibration data of the bone information (bone density) is differentiated and stored in the storage unit 320.

The first data obtaining unit 102 obtains the calibration data of the bone information (bone density) from the calibration data of the first bone information and the calibration data of the second bone information and stores the calibration data of the bone information (bone density) in the storage unit 320.

In the present step, the first data obtaining unit 102 obtains the calibration data of a plurality of pieces of bone information (bone density) according to the image processing setting (for example, whether the scattered ray reduction processing is ON or OFF) for the bone information calibration data and stores these in the storage unit 320. For the calibration data of the first bone information obtained in step S302 and the calibration data of the second bone information obtained in step S303, the first data obtaining unit 102 obtains the calibration data of the bone information (bone density) subjected to the scattered ray reduction processing and the calibration data of the bone information (bone density) not subjected to the scattered ray reduction processing and stores these in the storage unit 320.

The processing from step S306 to step S309 executed by the second data obtaining unit 103 is similar to the processing described in the first embodiment and will thus not be described.

### Step S310: Comparing the Comparison Information and Threshold

In step S310, the correction unit 104 obtains the comparison information indicating the overlapping ratio of the two irradiation field regions 402 and 404 by comparing the irradiation field region 402 obtained by imaging the QC phantom and the irradiation field region 404 obtained by imaging the object (patient). The correction unit 104 obtains the irradiation field region 402 obtained in step S304 and the irradiation field region 404 obtained in step S309 from the storage unit 320, compares the two irradiation field regions 402 and 404, and obtains the comparison information indicating the overlapping ratio of the two irradiation field regions 402 and 404.

The correction unit 104 compares the comparison information M₁ obtained using Mathematical Formula 4 and the threshold (804). Also, the correction unit 104 compares the comparison information M₂ obtained using Mathematical Formula 4 and the threshold (806). The threshold of the comparison information is a value set on the basis of the variation amount in the bone information as illustrated in FIG. 8(8A and 8B). In the present step, the threshold set based on the variation amount in the bone information is, in the preprocessing of the step S305, set the different thresholds depending on the setting of the image processing (whether the scattered ray reduction processing is ON or OFF, for example).

When the first comparison information (M₁) is equal to or greater than the first threshold (804) and the second comparison information (M₂) is equal to or greater than the second threshold (806) (a match in step S310), the correction unit 104 proceeds the processing to step S311.

### Step S311: Obtaining the Bone Information (Bone Density)

In step S311, the correction unit 104 executes processing similar to that from step S501 to step S505 to obtain the temporary bone information (bone density) of the object (patient). The correction unit 104 obtains corrected bone information (bone density) densA using Mathematical Formula 5 using the calibration values (bone information calibration data) a₀ and a₁ obtained in step S506 on the temporary bone information (bone density) dens obtained using Mathematical Formula 3.

In the first to fourth embodiments, in the example of comparing the first imaging condition and the second imaging condition described above, comparison information indicating the overlapping ratio of irradiation field regions obtained in steps S304 and S309 is used. However, in another example of comparing an imaging condition, as the radiation generation condition, the tube current, the irradiation duration, the tube voltage, and the like may be compared, or the irradiation angle at the radiation sensor 202, the SID, the grid type or presence, and similar geometric conditions may be compared.

According to the techniques disclosed in the present specification, the bone information obtaining accuracy can be improved.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2022-154001, filed Sep. 27, 2022, which is hereby incorporated by reference herein in its entirety.

What is claimed is:

1. An information processing apparatus comprising:
a first obtaining unit configured to, using data obtained by imaging a first object with known bone information via radiation irradiation based on a first imaging condition, obtain calibration data of the bone information; and
a correction unit configured to, using the calibration data, correct bone information of a second object different from the first object in a case where a comparison result of the first imaging condition and a second imaging condition satisfies a predetermined condition, the bone information of the second object being obtained using data obtained by imaging the second object via radiation irradiation based on the second imaging condition.

2. The information processing apparatus according to claim 1, wherein the first obtaining unit
obtains calibration data of first bone information using data obtained by imaging the first object via radiation of a first energy,
obtains calibration data of second bone information using data obtained by imaging the first object via radiation of a second energy higher than the first energy, and
obtains a first irradiation field region of radiation irradiated on a basis of the first imaging condition using the calibration data of the first bone information or the calibration data of the second bone information.

3. The information processing apparatus according to claim 2, wherein the first obtaining unit uses the calibration data of the first bone information or the calibration data of the second bone information to obtain a bone region included in the first object and a background region not including the bone region.

4. The information processing apparatus according to claim 3, wherein the first obtaining unit
obtains a bone region average value based on a difference between the calibration data of the second bone information and the calibration data of the first bone information in the bone region,
obtains a background region average value based on a difference between the calibration data of the second bone information and the calibration data of the first bone information in the background region, and
obtains the calibration data of the bone information for converting bone information of a bone region of the first object obtained from a difference between the background region average value and the bone region average value into the known bone information.

5. The information processing apparatus according to claim 4, wherein the first obtaining unit obtains the calibration data of the bone information via least squares approximation using bone information of a bone region of the first object and the known bone information.

6. The information processing apparatus according to claim 2, further comprising an image processing unit,
wherein the image processing unit executes image processing for reducing a scattered ray component included in the calibration data of the first bone information and the calibration data of the second bone information, and
the first obtaining unit obtains the calibration data of the first bone information with the scattered ray component reduced and the calibration data of the second bone information with the scattered ray component reduced.

7. The information processing apparatus according to claim 2, further comprising a second obtaining unit configured to
obtain first bone information using data obtained by imaging the second object via radiation of a first energy,
obtain second bone information using data obtained by imaging the second object via radiation of a second energy higher than the first energy, and
obtain a second irradiation field region of radiation irradiated on a basis of the second imaging condition using the first bone information or the second bone information.

8. The information processing apparatus according to claim 7, wherein the correction unit uses the first bone information or the second bone information to obtain a bone region included in the second object and a background region not including the bone region.

9. The information processing apparatus according to claim 8, wherein the correction unit
obtains a bone region average value based on a difference between the second bone information and the first bone information in the bone region,
obtains a background region average value based on a difference between the second bone information and the first bone information in the background region, and
obtains bone information in a bone region of the second object from a difference between the background region average value and the bone region average value.

10. The information processing apparatus according to claim 7, wherein the correction unit obtains an overlapping ratio of the first irradiation field region and the second irradiation field region as comparison information.

11. The information processing apparatus according to claim 10, wherein the correction unit
obtains first comparison information from an area ratio for the first irradiation field region obtained using an area of a first no-match region that is not overlapped with the second irradiation field region and an area of the first irradiation field region, and
obtains second comparison information from an area ratio for the second irradiation field region obtained using an area of a second no-match region that is not overlapped with the first irradiation field region and an area of the first irradiation field region.

12. The information processing apparatus according to claim 11, wherein the correction unit obtains a threshold based on a variation amount in bone information according to a degree of opening of a collimator,
obtains, as a first threshold, the comparison information of when a variation amount in bone information obtained when imaging while a degree of opening of the collimator is changed in a closed direction is equal to or greater than a specific variation amount, and
obtains, as a second threshold, the comparison information of when a variation amount in bone information obtained when imaging while a degree of opening of the collimator is changed in an open direction is equal to or greater than a specific variation amount.

13. The information processing apparatus according to claim 12, wherein, when the first comparison information is equal to or greater than the first threshold and the second comparison information is equal to or greater than the second threshold, the correction unit corrects the bone information of the second object using the calibration data.

14. The information processing apparatus according to claim 10, wherein the first obtaining unit obtains the calibration data of the bone information for a plurality of irradiation field regions with different sizes obtained from images from a plurality of instances of imaging,
the correction unit corrects the bone information using the calibration data of the bone information obtained for an irradiation field region with the highest comparison information from among the plurality of irradiation field regions obtained via comparison of the comparison information using the plurality of irradiation field regions and the second irradiation field region, or
the correction unit obtains a plurality of pieces of bone information using calibration data of bone information obtained for a plurality of irradiation field regions selected in order from highest of the comparison information, and
obtains bone information obtained via interpolation of the plurality of pieces of bone information using weighting according to the comparison information.

15. The information processing apparatus according to claim 12, wherein the second obtaining unit
obtains a second irradiation field region of radiation irradiated using the second imaging condition using first bone information obtained from data from imaging the second object via radiation of a first energy, and
when the first comparison information is less than the first threshold or the second comparison information is less than the second threshold, the first comparison information and the second comparison information being obtained from the first irradiation field region and the second irradiation field region, the correction unit stops imaging.

16. The information processing apparatus according to claim 15, further comprising a display control unit configured to cause a display unit to display a comparison result of the correction unit,
wherein the display control unit causes the display unit to display a message notify of the comparison result when the first comparison information is less than the first threshold or the second comparison information is less than the second threshold.

17. The information processing apparatus according to claim 16, wherein the display control unit
causes the display unit to display, in the message for confirming whether or not to perform re-imaging, an instruction input interface for instructing to perform re-imaging, an instruction input interface for cancelling the re-imaging, and a selection menu for input a reason for re-imaging when instructing to perform the re-imaging, or
causes the display unit to display a confirmation input interface for inputting an instruction to cancel the imaging.

18. A radiation imaging system comprising:
the information processing apparatus according to claim 1.

19. An information processing method comprising:
using data obtained by imaging a first object with known bone information via radiation irradiation based on a first imaging condition, obtaining calibration data of the bone information; and
using the calibration data, correcting bone information of a second object different from the first object in a case where a comparison result of the first imaging condition and a second imaging condition satisfies a predetermined condition, the bone information of the second object being obtained using data obtained by imaging the second object via radiation irradiation based on the second imaging condition

20. A non-transitory computer-readable storage medium storing a program for causing a computer to execute the method according to claim 19.
